(19) United States
(12) Patent Application Publication
(10) Pub. No.: US 2021/0378773 A1
(43) Pub. Date: Dec. 9, 2021

(54) STERILE INTERFACE ASSEMBLY FOR SURGICAL INSTRUMENTS SUCH AS FOR USE IN ROBOTIC SURGICAL SYSTEMS

(71) Applicant: Covidien LP, Mansfield, MA (US)

(72) Inventor: Zachary S. Heiliger, Nederland, CO (US)

(21) Appl. No.: 16/896,471

(22) Filed: Jun. 9, 2020

(51) Int. Cl. A61B 46/10; A61B 34/30; A61B 18/14

(52) U.S. Cl. CPC A61B 46/10; A61B 2017/00486; A61B 18/1442; A61B 34/30

(57) ABSTRACT

A sterile interface assembly configured to operably engage a robotic surgical instrument includes an adapter, a surgical drape, and a clip. The adapter includes a body having a first end portion and a second end portion, inputs disposed at the first end portion of the body, outputs disposed at the second end portion of the body, connectors coupling each of the inputs with a corresponding one of the outputs, and a seal disposed within the body and configured to establish a fluid-tight seal with an interior surface of the body and about the connectors. The surgical drape defines a sleeve of material having a proximal end portion and a distal end portion. The proximal end portion is attached to an exterior of the body of the adapter. The clip is configured to engage the distal end portion of the surgical drape to a shaft.

### FIELD

The present disclosure relates to surgical instruments and, more specifically, to a sterile interface assembly for surgical instruments such as, for example, for use in robotic surgical systems.

### BACKGROUND

Robotic surgical systems are increasingly utilized in various different surgical procedures. Some robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument.

The surgical instruments or portions thereof may be configured as single-use instruments or portions that are discarded after use, or may be configured as reusable instruments or portions that are cleaned and sterilized between uses. Regardless of the configurations of the surgical instruments, the console and robotic arm are capital equipment configured for long-term, repeated use. The console and robotic arm may be protected by a sterile barrier during use and/or wiped clean after use to ensure cleanliness for subsequent uses.

### SUMMARY

As used herein, the term “distal” refers to the portion that is being described which is further from an operator (whether a human surgeon or a surgical robot), while the term “proximal” refers to the portion that is being described which is closer to the operator. The terms “about,” substantially,” and the like, as utilized herein, are meant to account for manufacturing, material, environmental, use, and/or measurement tolerances and variations, and in any event may encompass differences of up to 10%.

Thus aspects and features of the present disclosure provide a sterile interface assembly to, for example, maintain sterility of a surgical environment and/or prevent contamination of capital equipment and other surgical instruments or portions thereof disposed proximally of the sterile interface assembly. To the extent consistent, any of the aspects described herein may be used in conjunction with any or all of the other aspects described herein.

Provided in accordance with aspects of the present disclosure is a sterile interface assembly configured to operably engage a robotic surgical instrument includes an adapter, a surgical drape, and a clip. The adapter includes a body having a first end portion and a second end portion, inputs disposed at the first end portion of the body, outputs disposed at the second end portion of the body, connectors coupling each of the inputs with a corresponding one of the outputs, and a seal disposed within the body and configured to establish a fluid-tight seal with an interior surface of the body and about the connectors. The surgical drape defines a sleeve of material having a proximal end portion and a distal end portion. The proximal end portion is attached to an exterior of the body of the adapter. The clip is configured to engage the distal end portion of the surgical drape to a shaft.

In aspects, the proximal end portion of the sleeve of material is sealingly attached to the exterior of the body of the adapter about a perimeter thereof. Additionally or alternatively, the clip is configured to sealingly engage the distal end portion of the surgical drape to a shaft.

In aspects, the body of the adapter includes at least one latching component configured to enable engagement of the body of the adapter with a housing.

In aspects, a second adapter is provided. The second adapter may include a body having a first end portion and a second end portion, inputs disposed at the first end portion of the body, outputs disposed at the second end portion of the body, and connectors coupling each of the inputs with a corresponding one of the outputs. The body of the second adapter may be configured to engage the body of the adapter such that the outputs of the second adapter are operably coupled to the inputs of the adapter.

In aspects, the inputs of the second adapter are configured to operably couple to outputs of a robotic surgical system. Alternatively or additionally, the body of the adapter includes at least one second latching component configured to enable engagement of the body of the adapter with the body of the second adapter.

In aspects, the surgical drape is substantially transparent.

### BRIEF DESCRIPTION OF THE DRAWINGS

Various aspects and features of the present disclosure are described hereinbelow with reference to the drawings wherein:

FIG. 1 is a perspective view of a surgical instrument in accordance with the present disclosure configured for mounting on a robotic arm of a robotic surgical system;

FIG. 2 is a rear perspective view of a proximal portion of the surgical instrument of FIG. 1 with an outer housing removed;

FIG. 3 is a schematic illustration of an exemplary robotic surgical system configured to releasably receive the surgical instrument of FIG. 1;

FIG. 4A is a perspective view of a sterile interface assembly mounted on a proximal end portion of the surgical instrument of FIG. 1 and including a proximal adapter;

FIG. 4B is a perspective view of the sterile interface assembly of FIG. 4A mounted on a proximal end portion of the surgical instrument of FIG. 1 without the proximal adapter;

FIG. 5 is an exploded, perspective view of the sterile interface assembly of FIG. 4A including the proximal adapter and a proximal portion of the surgical instrument of FIG. 1;

FIG. 6 is a perspective view of the proximal adapter of FIG. 4A;

FIG. 7 is a longitudinal, cross-sectional view taken along section line “7-7” of FIG. 4B; and

FIGS. 8A-8C illustrate engagement of the sterile interface assembly of FIG. 4A with the proximal portion of the surgical instrument of FIG. 1.

### DETAILED DESCRIPTION

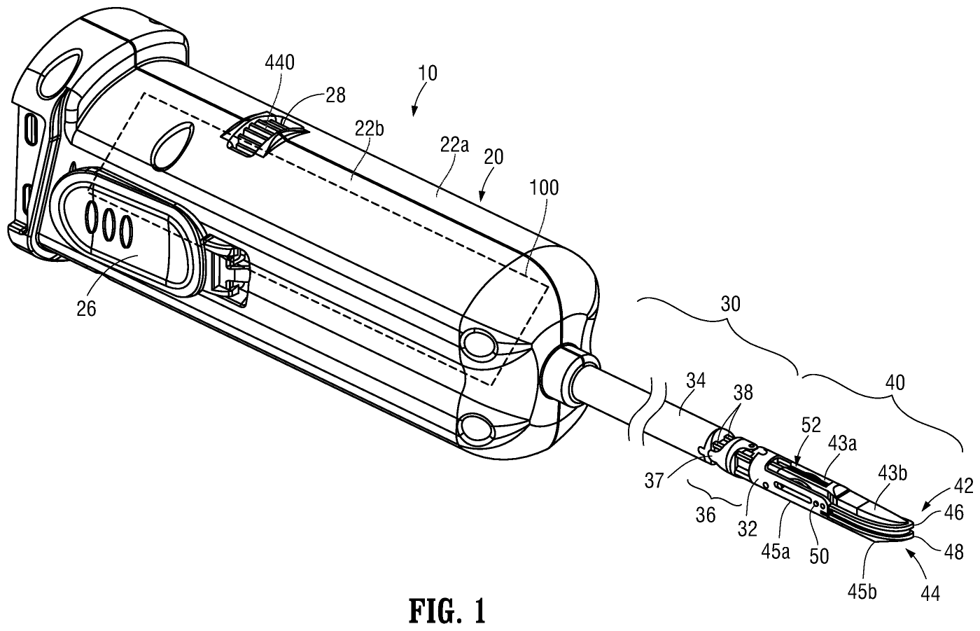

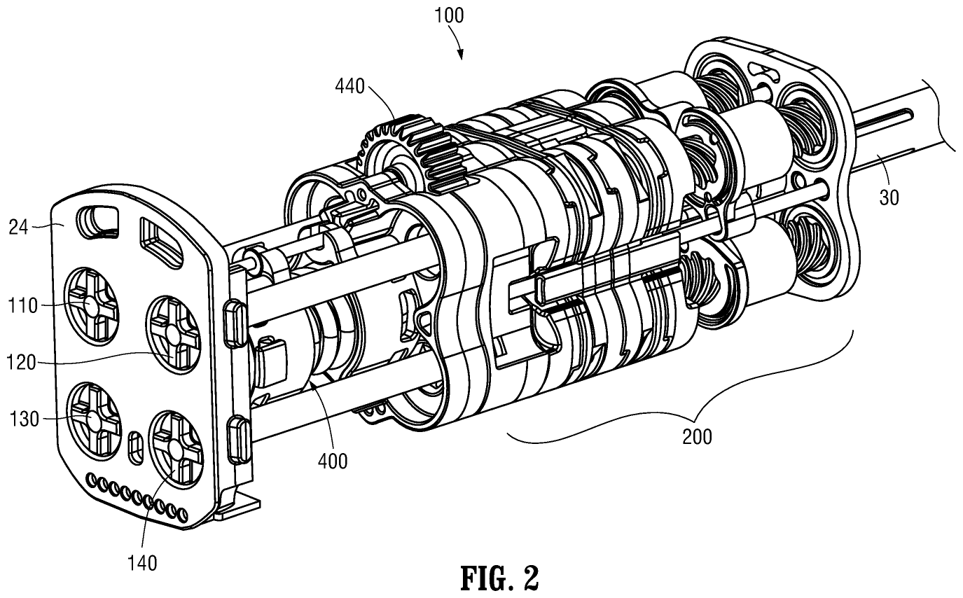

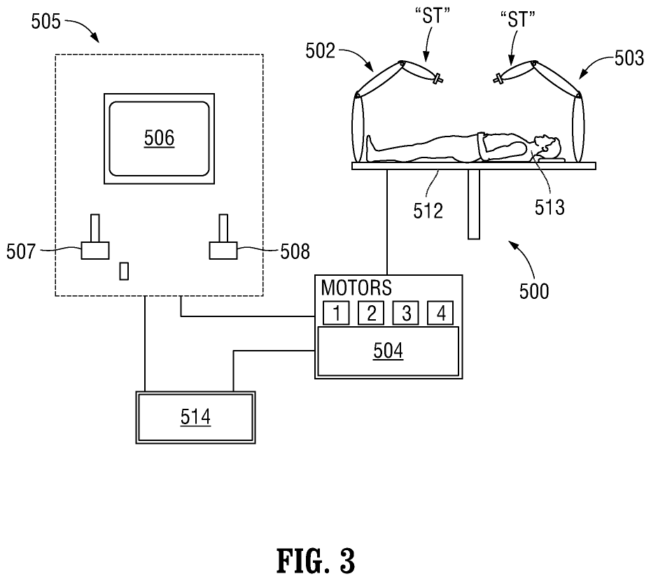

Referring to FIGS. 1 and 2, a surgical instrument 10 provided in accordance with the present disclosure generally includes a housing 20, a shaft 30 extending distally from housing 20, an end effector assembly 40 extending distally from shaft 30, and an actuation assembly 100 disposed within housing 20 and operably associated with shaft 30 and end effector assembly 40. Instrument 10 is detailed herein as an articulating electrosurgical forceps configured for use with a robotic surgical system, e.g., robotic surgical system 500 (FIG. 3). However, the aspects and features of instrument 10 provided in accordance with the present disclosure, detailed below, are equally applicable for use with other suitable surgical instruments and/or in other suitable surgical systems.

Housing 20 of instrument 10 includes first and second body portion 22a, 22b and a proximal face plate 24 (FIG. 2) that cooperate to enclose actuation assembly 100 therein. Proximal face plate 24 includes apertures defined therein through which inputs 110-140 of actuation assembly 100 extend. A pair of latch levers 26 (only one of which is illustrated in FIG. 1) extends outwardly from opposing sides of housing 20 and enables releasable engagement (directly or indirectly) of housing 20 with a robotic arm of a surgical system, e.g., robotic surgical system 500 (FIG. 3). An aperture 28 defined through housing 20 permits thumbwheel 440 to extend therethrough to enable manual manipulation of thumbwheel 440 from the exterior of housing 20 to permit manual opening and closing of end effector assembly 40.

Shaft 30 of instrument 10 includes a distal segment 32, a proximal segment 34, and an articulating section 36 disposed between the distal and proximal segments 32, 34, respectively. Articulating section 36 includes one or more articulating components 37, e.g., links, joints, etc. A plurality of articulation cables 38, e.g., four (4) articulation cables, or other suitable actuators, extends through articulating section 36. More specifically, articulation cables 38 are operably coupled to distal segment 32 of shaft 30 at the distal ends thereof and extend proximally from distal segment 32 of shaft 30, through articulating section 36 of shaft 30 and proximal segment 34 of shaft 30, and into housing 20, wherein articulation cables 38 operably couple with an articulation assembly 200 of actuation assembly 100 to enable selective articulation of distal segment 32 (and, thus end effector assembly 40) relative to proximal segment 34 and housing 20, e.g., about at least two axes of articulation (yaw and pitch articulation, for example). Articulation cables 38 are arranged in a generally rectangular configuration, although other suitable configurations are also contemplated.

With respect to articulation of end effector assembly 40 relative to proximal segment 34 of shaft 30, actuation of articulation cables 38 is effected in pairs. More specifically, in order to pitch end effector assembly 40, the upper pair of cables 38 is actuated in a similar manner while the lower pair of cables 38 is actuated in a similar manner relative to one another but an opposite manner relative to the upper pair of cables 38. With respect to yaw articulation, the right pair of cables 38 is actuated in a similar manner while the left pair of cables 38 is actuated in a similar manner relative to one another but an opposite manner relative to the right pair of cables 38.

End effector assembly 40 includes first and second jaw members 42, 44, respectively. Each jaw member 42, 44 includes a proximal flange portion 43a, 45a and a distal body portion 43b, 45b, respectively. Distal body portions 43b, 45b define opposed tissue-contacting surfaces 46, 48, respectively. Proximal flange portions 43a, 45a are pivotably coupled to one another about a pivot 50 and are operably coupled to one another via a cam-slot assembly 52 including a cam pin slidably received within cam slots defined within the proximal flange portion 43a, 45a of at least one of the jaw members 42, 44, respectively, to enable pivoting of jaw member 42 relative to jaw member 44 and distal segment 32 of shaft 30 between a spaced-apart position (e.g., an open position of end effector assembly 40) and an approximated position (e.g. a closed position of end effector assembly 40) for grasping tissue between tissue-contacting surfaces 46, 48. As an alternative to this unilateral configuration, a bilateral configuration may be provided whereby both jaw members 42, 44 are pivotable relative to one another and distal segment 32 of shaft 30.

In some configurations, longitudinally-extending knife channels (not shown) are defined through tissue-contacting surfaces 46, 48, respectively, of jaw members 42, 44. In such configurations, a knife assembly (not shown) is provided including a selectively advanceable knife that enables cutting of tissue grasped between tissue-contacting surfaces 46, 48 of jaw members 42, 44, respectively. A knife drive assembly (not shown) of actuation assembly 100 provides for selective actuation of the knife assembly to reciprocate the knife between jaw members 42, 44 to cut tissue grasped between tissue-contacting surfaces 46, 48. Alternatively or additionally, energy-based cutting may be provided via a static and/or dynamic cutting member, e.g., a cutting electrode, thermal cutting element, knife, etc.

Continuing with reference to FIGS. 1 and 2, a drive rod (not shown) is operably coupled to cam-slot assembly 52 of end effector assembly 40, e.g., engaged with the cam pin thereof, such that longitudinal actuation of drive rod pivots jaw member 42 relative to jaw member 44 between the spaced-apart and approximated positions. More specifically, urging the drive rod proximally pivots jaw member 42 relative to jaw member 44 towards the approximated position while urging the drive rod distally pivots jaw member 42 relative to jaw member 44 towards the spaced-apart position. However, other suitable mechanisms and/or configurations for pivoting jaw member 42 relative to jaw member 44 between the spaced-apart and approximated positions in response to selective actuation of a drive rod are also contemplated. The drive rod extends proximally from end effector assembly 40 through shaft 30 and into housing 20 wherein the drive rod is operably coupled with a jaw drive assembly 400 of actuation assembly 100 (FIG. 2) to enable selective actuation of end effector assembly 40 to grasp tissue therebetween and apply a closure force within an appropriate jaw closure force range.

Tissue-contacting surfaces 46, 48 of jaw members 42, 44, respectively, are at least partially formed from an electrically conductive material and are energizable to different potentials to enable the conduction of electrical energy through tissue grasped therebetween, although tissue-contacting surfaces 46, 48 may alternatively be configured to supply any suitable energy, e.g., thermal, microwave, light, ultrasonic, etc., through tissue grasped therebetween for energy-based tissue treatment. Instrument 10 defines a conductive pathway (not shown) through housing 20 and shaft 30 to end effector assembly 40 that may include lead wires, contacts, and/or electrically-conductive components to enable electrical connection of tissue-contacting surfaces 46, 48 of jaw members 42, 44, respectively, to an energy source (not shown), e.g., an electrosurgical generator via an electrosurgical cable extending therebetween, for supplying energy to tissue-contacting surfaces 46, 48 to treat, e.g., seal, tissue grasped between tissue-contacting surfaces 46, 48.

As noted above, actuation assembly 100 is disposed within housing 20 and includes an articulation assembly 200, a knife drive assembly (not shown), and a jaw drive assembly 400. Articulation assembly 200 is operably coupled between first and second inputs 110, 120, respectively, of actuation assembly 100 and articulation cables 38 (FIG. 1) such that, upon receipt of appropriate rotational inputs into first and/or second inputs 110, 120, articulation assembly 200 manipulates cables 38 (FIG. 1) to articulate end effector assembly 40 in a desired direction, e.g., to pitch and/or yaw end effector assembly 40. The knife drive assembly is operably coupled between third input 130 of actuation assembly 100 and the knife assembly such that, upon receipt of appropriate rotational input into third input 130, the knife drive assembly manipulates the knife assembly to reciprocate the knife blade 68 between jaw members 42, 44 to cut tissue grasped between tissue-contacting surfaces 46, 48. Jaw drive assembly 400 is operably coupled between fourth input 140 of actuation assembly 100 and the drive rod such that, upon receipt of appropriate rotational input into fourth input 140, jaw drive assembly 400 pivots jaw members 42, 44 between the spaced-apart and approximated positions to grasp tissue therebetween and apply a closure force within an appropriate closure force range.

Actuation assembly 100 is configured to operably interface with a robotic surgical system 500 (FIG. 3) when instrument 10 is mounted on robotic surgical system 500 (FIG. 3), to enable robotic operation of actuation assembly 100 to provide the above-detailed functionality. That is, robotic surgical system 500 (FIG. 3) selectively provides rotational inputs to inputs 110-140 of actuation assembly 100 to articulate end effector assembly 40, grasp tissue between jaw members 42, 44, and/or cut tissue grasped between jaw members 42, 44. However, it is also contemplated that actuation assembly 100 be configured to interface with any other suitable surgical system, e.g., a manual surgical handle, a powered surgical handle, etc. For the purposes herein, robotic surgical system 500 (FIG. 3) is generally described.

Turning to FIG. 3, robotic surgical system 500 is configured for use in accordance with the present disclosure. Aspects and features of robotic surgical system 500 not germane to the understanding of the present disclosure are omitted to avoid obscuring the aspects and features of the present disclosure in unnecessary detail.

Robotic surgical system 500 generally includes a plurality of robot arms 502, 503; a control device 504; and an operating console 505 coupled with control device 504. Operating console 505 may include a display device 506, which may be set up in particular to display three-dimensional images; and manual input devices 507, 508, by means of which a person, e.g., a surgeon, may be able to telemanipulate robot arms 502, 503 in a first operating mode. Robotic surgical system 500 may be configured for use on a patient 513 lying on a patient table 512 to be treated in a minimally invasive manner. Robotic surgical system 500 may further include a database 514, in particular coupled to control device 504, in which are stored, for example, pre-operative data from patient 513 and/or anatomical atlases.

Each of the robot arms 502, 503 may include a plurality of members, which are connected through joints, and a mounted device which may be, for example, a surgical tool “ST.” One or more of the surgical tools “ST” may be instrument 5 (FIG. 1), thus providing such functionality on a robotic surgical system 500.

Robot arms 502, 503 may be driven by electric drives, e.g., motors, connected to control device 504. Control device 504, e.g., a computer, may be configured to activate the motors, in particular by means of a computer program, in such a way that robot arms 502, 503, and, thus, their mounted surgical tools “ST” execute a desired movement and/or function according to a corresponding input from manual input devices 507, 508, respectively. Control device 504 may also be configured in such a way that it regulates the movement of robot arms 502, 503 and/or of the motors.

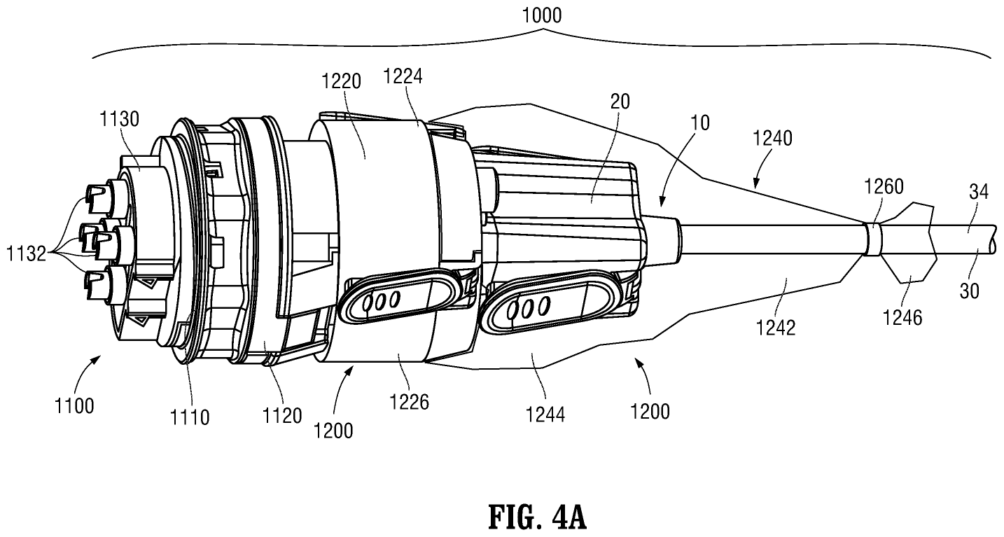

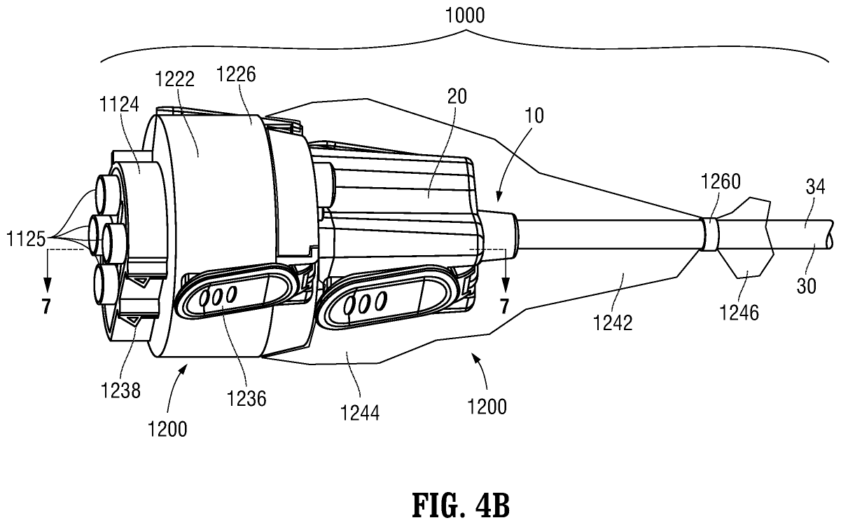

Turning to FIGS. 4A-7, in order to maintain sterility of a surgical environment and/or prevent contamination of robotic surgical system 500 (FIG. 3), a sterile interface assembly 1000 is provided for operably coupling surgical instrument 10 (or any other suitable surgical instrument) with a robotic surgical system, e.g., robotic surgical system 500 (FIG. 3). Sterile interface assembly 1000 includes a proximal adapter 1100 and a seal module 1200 including a distal adapter 1220, a sterile drape 1240, and a clip 1260. Seal module 1200 is configured to sealingly operably engage housing 20 and proximal segment 34 of shaft 30 of instrument 10, while proximal adapter 1100 is configured to connect distal adapter 1220 of seal module 1200 with robotic surgical system 500 (FIG. 3). In some configurations, proximal adapter 1100 is omitted and distal adapter 1220 of seal module 1200 is configured to connect directly with robotic surgical system 500 (FIG. 3). In other configurations, proximal adapter 1100 is utilized but is not part of sterile interface assembly 1000. Further, although the adapters 1100, 1220 are detailed herein as proximal and distal, respectively, it is contemplated that other relatively orientations are also contemplated, e.g., wherein the adapter 1220 is more-proximal and the adapter 1100 is more distal, wherein the adapters 1100, 1220 are laterally disposed, etc.

Sterile interface assembly 1000 may be configured for single-use; that is, where sterile interface assembly 1000 is discarded after use or sent to a manufacturer for reprocessing. Alternatively, sterile interface assembly 1000 may be reusable; that is, capable of being cleaned and/or sterilized for repeated use by the end-user. Further still, sterile interface 1000 may be partially-single-use and partially-reusable; that is, where proximal adapter 1100 is configured as a cleanable/sterilizable, reusable component, while seal module 1200 is configured as a single-use, disposable/reprocessable component, or vice versa. In any of the above configurations, proximal adapter 1100 and seal module 1200 may be configured to releasably engage one another to facilitate disposal/reprocessing of any single-use components and cleaning and/or sterilization of any reusable components.

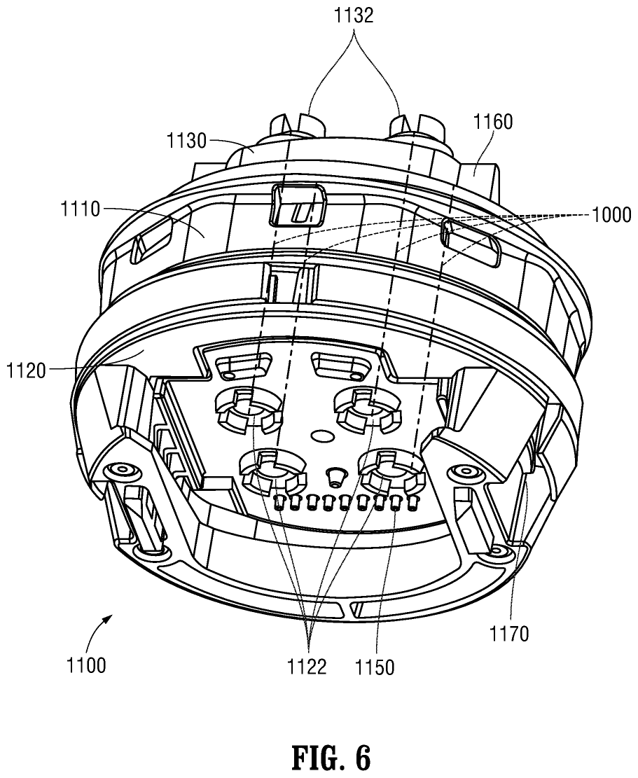

With reference to FIG. 6, proximal adapter 1100 includes a body 1110 defining a proximal face portion 1120 and a distal face portion 1130. A plurality of inputs 1122 are disposed at proximal face portion 1120, a plurality of outputs 1132 are disposed at distal face portion 1130, and a plurality of connectors 1140 extend through body 1110 to operably interconnect corresponding pairs of inputs 1122 and outputs 1132 with one another. Inputs 1122 and outputs 1132 are illustrated as pronged rotational inputs and outputs; however, inputs 1122 and outputs 1132 may alternatively include any other suitable combination of similar or different configurations such as, for example, complementary, male, female, and/or hermaphroditic inputs/outputs for providing rotational, translational, or combination rotational and translation motion. Connectors 1140 may be configured for providing matching inputs and outputs, may be configured to amplify or attenuate the outputs as compared to the inputs, may be configured to convert one mode of input into a different mode of output (e.g., rotation to translation or vice versa), or may be configured in any other suitable manner. Connectors 1140 may include one or more shafts, gears, pulleys, cables, etc. to operably interconnect the corresponding pairs of inputs 1122 and outputs 1132 with one another. Proximal adapter 1100 may further include electrical connectors (not shown) extending through body 1110 to electrically couple electrical inputs 1152 at proximal face portion 1120 with corresponding electrical outputs (not shown) at distal face portion 1130 thereof, e.g., for data transfer, communication, sensing, energy delivery, etc.

Body 1110 houses connectors 1140 therein. Body 1110 further includes, formed therewith or otherwise attached thereto, latching components 1160, 1170 disposed at distal face portion 1130 and proximal face portion 1120, respectively. Latching components 1160, 1170 enable releasable engagement of proximal adapter 1100 with seal module 1200 and the robotic surgical system 500 (FIG. 3), e.g., via mechanical latching, snap-fit engagement, press-fit engagement, or other suitable engagement.

Referring to FIGS. 4A, 4B, 5, and 7, seal module 1200, as noted above, includes distal adapter 1220, sterile drape 1240, and clip 1260. Distal adapter 1220 includes a body 1222 defining a proximal face portion 1224 and a distal face portion 1226. A plurality of inputs 1225 are disposed at proximal face portion 1224, a plurality of outputs 1227 are disposed at distal face portion 1226, and a plurality of connectors 1230 extend through body 1222 to operably interconnect corresponding pairs of inputs 1225 and outputs 1227 with one another.

Inputs 1225 and outputs 1227 are illustrated as pronged rotational inputs and outputs; however, inputs 1225 and outputs 1227 may alternatively include any other suitable combination of similar or different configurations such as, for example, complementary, male, female, and/or hermaphroditic inputs/outputs for providing rotational, translational, or combination rotational and translation motion. Connectors 1230 may be configured for providing matching inputs and outputs, may be configured to amplify or attenuate the outputs as compared to the inputs, may be configured to convert one mode of input into a different mode of output (e.g., rotation to translation or vice versa), or may be configured in any other suitable manner. Connectors 1230 may include one or more shafts, gears, pulleys, cables, etc. to operably interconnect the corresponding pairs of inputs 1225 and outputs 1227 with one another. Distal adapter 1220 may further include electrical connectors (not shown) extending through body 1222 to electrically couple electrical inputs (not shown) at proximal face portion 1224 with corresponding electrical outputs (not shown) at distal face portion 1226 thereof, e.g., for data transfer, communication, sensing, energy delivery, etc.

Body 1222 houses connectors 1230 therein. Body 1220 further includes a seal 1234 disposed therein and configured to establish a fluid-tight seal within an interior of body 1222 about connectors 1230 (and the electrically connectors, where provided). Seal 1234 may be a single-piece seal, a multi-piece seal, and injectable material forming a seal, or any other suitable seal. Seal 1234 establishes a fluid-tight seal within the interior of body 1222 and about connectors 1230 to inhibit proximal transmission of fluid, e.g., blood, water, saline, surgical fluids such as insufflation fluid, etc., across seal 1234. Seal 1234 maintains the fluid-tight seal while allowing rotation (or other actuation) of connectors 1230.

Body 1222 additionally includes, formed therewith or otherwise attached thereto, latching components 1236, 1238 disposed towards distal face portion 1226 and proximal face portion 1224, respectively. Latching components 1236, 1238 enable releasable engagement of distal adapter 1100 with housing 20 of instrument 10, e.g., via mechanical latching (such as with latch levers (as illustrated)), snap-fit engagement, press-fit engagement, or other suitable engagement, and with proximal adapter 1100 (via engagement of latching components 1160 with latching components 1238) or directly to the robotic surgical system 500 (FIG. 3).

Continuing with reference to FIGS. 4A, 4B, 5, and 7, sterile drape 1240 is configured as a sleeve of material 1242 that inhibits the passage of fluid, e.g., blood, water, saline, surgical fluids such as insufflation fluid, etc., therethrough. Sleeve of material 1242 may be substantially transparent to enable visualization therethrough and may be sufficiently pliable to enable external manipulation of any controls disposed on housing 20 of instrument 10 when sterile drape 1240 is disposed thereabout. A proximal end portion 1244 of sleeve of material 1242 is sealed about an exterior outer perimeter of body 1222 of distal adapter 1220, e.g., via an adhesive, clip, overmolding, and/or other suitable engagement. In this manner, fluid, e.g., blood, water, saline, surgical fluids such as insufflation fluid, etc., within sleeve of material 1242 is inhibited from passing proximally between proximal end portion 1244 of sleeve of material 1242 of sterile drape 1240 and body 1222 of distal adapter 1220.

A distal end portion 1246 of sleeve of material 1242 is configured to be engaged about a portion of proximal segment 34 of shaft 30 of instrument 10, e.g., via clip 1260, and, in some configurations, establishes a fluid-tight seal about proximal segment 34 of shaft 30. Thus, sleeve of material 1242 of surgical drape 1240 defines a sealed interior volume enclosing a portion of distal adapter 1240 as well as housing 20 of instrument 10 and a portion of proximal segment 34 of shaft 30 of instrument 10. This sealed interior volume, together with the sealing of the interior of body 1222 of distal adapter 1220 via seal 1234, provides a sterile barrier to inhibit fluid, e.g., blood, water, saline, surgical fluids such as insufflation fluid, etc., from passing therebeyond.

Clip 1260 may be configured as a hinged clip, c-clip, elastomeric ring, or other suitable clip configured to engage (and, in some configurations, sealingly engage), distal end portion 1246 of sleeve of material 1242 about proximal segment 34 of shaft 30 of instrument 10.

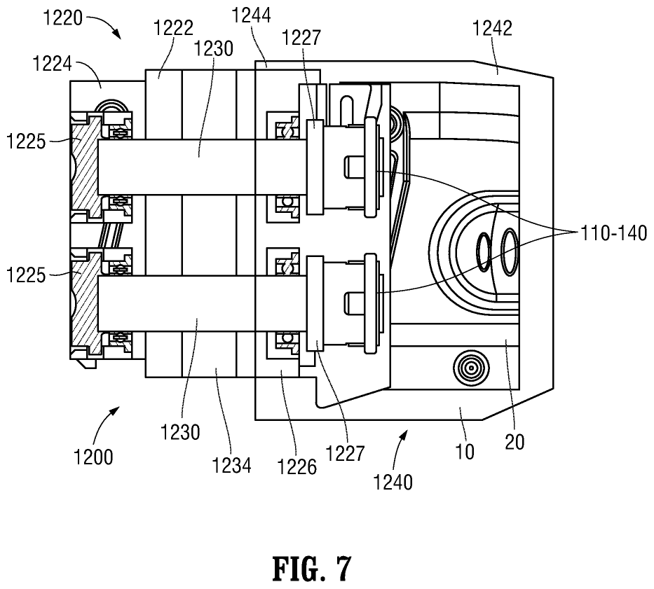

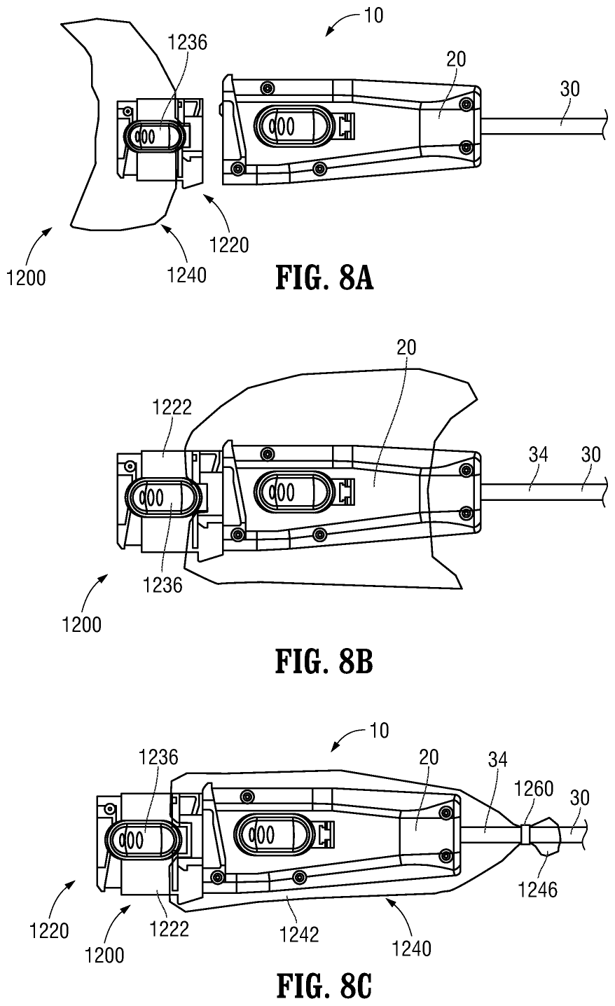

Turning to FIGS. 8A-8C, in conjunction with FIGS. 4B, 5, and 7, and initially to FIG. 8A, in order to assemble seal module 1200 about instrument 10, distal adapter 1220 is approximated relative to a proximal portion of housing 20 and proximal face plate 24, e.g., longitudinally, transversely, rotationally, pivotally, or in any other suitable manner, such that distal face portion 1226 of distal adapter 1220 substantially abuts proximal face plate 24 of housing 20 with latching components 1236 engaging corresponding features, e.g., recesses, defined within housing 20 to thereby releasably engage distal adapter 1220 and housing 20 with one another with outputs 1227 of distal adapter 1220 operably engaged with inputs 110-140 (see FIG. 2) of instrument 10 (see also FIG. 7).

Once the above engagement is achieved, as shown in FIG. 8B, distal end portion 1246 of sleeve of material 1242 of surgical drape 1240 is pulled distally over housing 20 and a portion of proximal segment 34 of shaft 30 of instrument 10. Thereafter, as shown in FIG. 8C, clip 1260 is attached to engage (and, in some configurations, sealingly engage), distal end portion 1246 of sleeve of material 1242 about proximal segment 34 of shaft 30 of instrument 10.

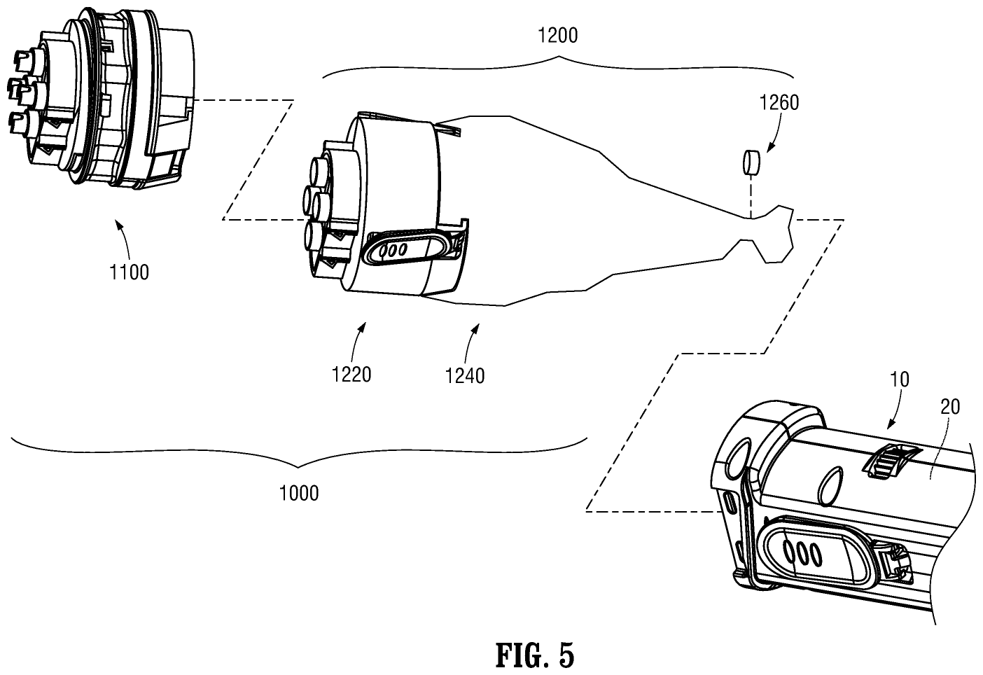

Referring also to FIGS. 4A and 5, thereafter, if utilized, proximal adapter 1100 may be engaged with distal adapter 1220 of seal module 1200, e.g., via engagement of latching components 1160, 1238 with one another, to thereby operably, e.g., rotationally, couple outputs 1132 of proximal adapter 1100 with inputs 1225 of distal adapter 1220, thereby operably coupling inputs 1122 of proximal adapter 1100 with inputs 110-140 (FIG. 2) of instrument 10. As such, upon engagement of proximal adapter 1100 with the robotic surgical system 500 (FIG. 3), inputs provided by the robotic surgical system 500 (FIG. 3) to inputs 1122 of proximal adapter 1100 are provided to corresponding inputs 110-140 (FIG. 2) of instrument 10. Alternatively, as noted above, distal adapter 1220 may be engaged directly to the robotic surgical system 500 (FIG. 3) to operable in a similar manner except without proximal adapter 1100.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A sterile interface assembly configured to operably engage a robotic surgical instrument, the assembly comprising:
an adapter, including:
a body having a first end portion and a second end portion;
inputs disposed at the first end portion of the body;
outputs disposed at the second end portion of the body;
connectors coupling each of the inputs with a corresponding one of the outputs; and
a seal disposed within the body and configured to establish a fluid-tight seal with an interior surface of the body and about the connectors;
a surgical drape defining a sleeve of material having a proximal end portion and a distal end portion, the proximal end portion attached to an exterior of the body of the adapter; and
a clip configured to engage the distal end portion of the surgical drape to a shaft.

2. The sterile interface assembly according to claim 1, wherein the proximal end portion of the sleeve of material is sealingly attached to the exterior of the body of the adapter about a perimeter thereof.

3. The sterile interface assembly according to claim 1, wherein the clip is configured to sealingly engage the distal end portion of the surgical drape to a shaft.

4. The sterile interface assembly according to claim 1, wherein the body of the adapter includes at least one latching component configured to enable engagement of the body of the adapter with a housing.

5. The sterile interface assembly according to claim 1, further comprising:
a second adapter, including:
a body having a first end portion and a second end portion;
inputs disposed at the first end portion of the body;
outputs disposed at the second end portion of the body; and
connectors coupling each of the inputs with a corresponding one of the outputs,
wherein the body of the second adapter is configured to engage the body of the adapter such that the outputs of the second adapter are operably coupled to the inputs of the adapter.

6. The sterile interface assembly according to claim 5, wherein the inputs of the second adapter are configured to operably couple to outputs of a robotic surgical system.

7. The sterile interface assembly according to claim 5, wherein the body of the adapter includes at least one second latching component configured to enable engagement of the body of the adapter with the body of the second adapter.

8. The sterile interface assembly according to claim 1, wherein the surgical drape is substantially transparent.
